(19) United States
(12) Patent Application Publication
(10) Pub. No.: US 2026/0002951 A1
(43) Pub. Date: Jan. 1, 2026

(54) METHOD FOR PROVIDING INFORMATION FOR BRAIN DOPING DIAGNOSIS

(71) Applicant: KOREA INSTITUTE OF SCIENCE AND TECHNOLOGY, Seoul (KR)

(72) Inventors: Junghyun SON, seoul (KR); Hyung Min Kim, Seoul (KR); Yoeseph Cho, Seoul (KR); Ji Hyeok Jeong, Seoul (KR); Dong Jin Sung, Seoul (KR)

(21) Appl. No.: 18/978,722

(22) Filed: Dec. 12, 2024

(30) Foreign Application Priority Data
Jun. 28, 2024 (KR) 10-2024-0085448

(51) Int. Cl. G01N 33/94; A61B 5/00; G01N 33/68

(52) U.S. Cl. CPC G01N 33/9433; G01N 33/6812; A61B 5/4064; G01N 2800/28

(57) ABSTRACT

The present disclosure relates to a biomarker for providing information on brain electrical stimulation, and a method for providing information for brain doping diagnosis (bD²) using the same. According to the present disclosure, there is provided a method for determining, using a urine sample, whether a subject has received brain electrical stimulation, which may be used as a means for establishing regulations and countermeasures for abuse of brain doping of which safety has not yet been confirmed.

### CROSS-REFERENCE TO RELATED APPLICATION

This application claims the benefit of Korean Patent Application No. 10-2024-0085448 filed on Jun. 28, 2024, in the Korean Intellectual Property Office, the entire disclosure of which is incorporated herein by reference for all purposes.

### BACKGROUND

### 1. Field of the Invention

The present disclosure relates to a biomarker for providing information on brain electrical stimulation, and a method for providing information for brain doping diagnosis using the same.

### 2. Description of the Related Art

Recently, as brain science technologies for which evidence is hardly found are appearing in the sports field, voices of concern are growing. A technology of improving physical, intellectual, and cognitive abilities by activating specific areas by applying electrical stimulation to the brain is called ‘brain doping.’ A representative example may be ‘transcranial direct current stimulation (tDCS).’ The tDCS is a technology that causes the activity of nerve cells by running a weak direct current to the brain surface through electrodes on the scalp. As a method for treating brain diseases or for normalizing brain functions, when drug treatment is clinically limited or difficult to use, the tDCS has been used in combination with drug treatment. The effects of tDCS on sports performance are controversial, but in 2016, as a result of a brain stimulation experiment in which athletes from the United States Ski and Snowboard Association were involved, it was reported that a 70% increase in jumping power and an 80% increase in balance were shown. In addition, to this day, research results showing increased motor ability have been continuously reported not only in the general public but also in actual athletics, taekwondo, and volleyball players.

It is necessary to observe brain doping for a long time with deep interest. The World Anti-Doping Agency has defined that doping occurs when two of the following conditions are met: First, whether to have a potentially beneficial effect, second, whether to cause a potential health risk to an athlete, third, whether to violate the sportsmanship, and the like. First, study results show that tDCS technology has the potential to improve motor ability. The second condition remains controversial. The tDCS technology does not cause a serious threat to normal health, but no studies have been conducted on adverse effects from long-term use beyond acceptable intensities. In the case of the last condition, it is interpreted that the tDCS technology violates the sportsmanship of equality and fairness in that the tDCS technology temporarily improves motor ability without efforts.

A brain stimulation method has been used in clinical fields as treatment and rehabilitation methods for neurological diseases, and it is known that safety is secured within limited stimulation intensity and time. However, since there has yet been no research on effects on the human body when indiscriminately abused for improving athletic performance, the present inventors have continuously conducted research on brain doping diagnosis (bD²) in order to establish regulations and countermeasures for the possibility of abuse of brain stimulation for improving athletic performance in sports, and then completed the present disclosure to utilize the brain doping diagnosis as a brain doping test strategy.

### SUMMARY

Embodiments provide a biomarker capable of diagnosing or predicting brain electrical stimulation for a brain doping test, and a method for providing information for brain doping diagnosis using the biomarker.

However, aspects of the present disclosure are not limited to the aforementioned aspects, and other aspects which are not mentioned can be clearly understood to those skilled in the art from the following description.

According to an aspect, there is provided a biomarker composition for providing information on brain electrical stimulation including at least one indicator selected from the group consisting of a ratio of epinephrine to 3-methoxytyramine (EP/3-MT), a ratio of histamine to homocysteine (His/Hcy), a ratio of tyramine to homocysteine (TA/Hcy), a ratio of vanillylmandelic acid to 3-methoxytyramine (VMA/3-MT), a ratio of vanillylmandelic acid to 4-hydroxy-3-methoxy-phenylglycol (VMA/MHPG), a ratio of homocysteine to 3-methoxytyramine (Hcy/3-MT), a ratio of homocysteine to serotonin (Hcy/5-HT), a ratio of homocysteine to aspartic acid (Hcy/Asp), a ratio of tryptophan to epinephrine (Trp/EP), and a ratio of tyrosine to epinephrine (Tyr/EP), which are detected in a urine sample.

The biomarker of the present disclosure is used to predict whether there is a history of receiving electrical stimulation to the brain to improve motor ability, and may be used for a brain doping test.

Accordingly, the present disclosure provides a brain doping test method and the method may be used as a method for providing information for brain doping diagnosis.

As an embodiment of the present disclosure, the electrical stimulation may be performed before about one week, desirably 48 hours, more desirably 24 hours, 8 hours, 2 hours, or 1 hour based on a brain doping testing time point.

According to another aspect, there is provided a brain doping test method including the following steps:
(A) obtaining a urine sample from a subject;
(B) preparing a neurochemical profile by measuring the contents of neurotransmitters and metabolites thereof in the sample; and
(C) calculating one or more indicators selected from the group consisting of a ratio of epinephrine to 3-methoxytyramine (EP/3-MT), a ratio of histamine to homocysteine (His/Hcy), a ratio of tyramine to homocysteine (TA/Hcy), a ratio of vanillylmandelic acid to 3-methoxytyramine (VMA/3-MT), a ratio of vanillylmandelic acid to 4-hydroxy-3-methoxy-phenylglycol (VMA/MHPG), a ratio of homocysteine to 3-methoxytyramine (Hcy/3-MT), a ratio of homocysteine to serotonin (Hcy/5-HT), a ratio of homocysteine to aspartic acid (Hcy/Asp), a ratio of tryptophan to epinephrine (Trp/EP), and a ratio of tyrosine to epinephrine (Tyr/EP) from the profile.

As an embodiment of the present disclosure, the brain doping test method is a method for providing information on whether or not there is a history of receiving electrical stimulation to the brain to improve motor ability, and may provide information on whether the subject has received brain electrical stimulation within about one week, desirably within 48 hours, more desirably within 24 hours, 8 hours, or 2 hours based on a brain doping test time point.

As an embodiment of the present disclosure, the test method may further include the following step D after step C:
(D) determining that the subject has a history of receiving electrical stimulation to the brain or to have a high possibility of receiving electrical stimulation to the brain in the case of one or more of the following conditions (1) to (10).
(1) when the ratio of epinephrine to 3-methoxytyramine (EP/3-MT) is greater than 0.74
(2) when the ratio of histamine to homocysteine (His/Hcy) is greater than 3.27
(3) when the ratio of tyramine to homocysteine (TA/Hcy) is greater than 7.72
(4) when the ratio of vanillylmandelic acid to 3-methoxytyramine (VMA/3-MT) is greater than 182
(5) when the ratio of vanillylmandelic acid to 4-hydroxy-3-methoxy-phenylglycol (VMA/MHPG) is greater than 43.3
(6) when the ratio of homocysteine to 3-methoxytyramine (Hcy/3-MT) is less than 2.66
(7) when the ratio of homocysteine to serotonin (Hcy/5-HT) is less than 0.56
(8) when the ratio of homocysteine to aspartic acid (Hcy/Asp) is less than 0.29
(9) when the ratio of tryptophan to epinephrine (Trp/EP) is less than 663
(10) when the ratio of tyrosine to epinephrine (Tyr/EP) is less than 1036

According to embodiments, there is provided a method for determining, using a urine sample, whether a subject has received brain electrical stimulation, which may be used as a means for establishing regulations and countermeasures for abuse of brain doping of which safety has not yet been confirmed.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIGS. 1A and 1B show locations of stimulations for inducing brain doping through current stimulation (FIG. 1A), an actual electrical stimulation applied to an experimental group (upper graph in FIG. 1B), and a sham stimulation applied to a control group (lower graph in FIG. 1B);

FIG. 2 is a schematic diagram of an experiment;

FIG. 3 illustrates results of exercise evaluation of an experimental group (Stimulation) and a control group (Sham);

FIG. 4 illustrates results of measuring brain wave changes of Subjects 6 and 12 after electrical stimulation;

FIG. 5 illustrates results of average values obtained by measuring brain wave changes of all subjects after electrical stimulation;

FIG. 6 illustrates LC-MS/MS analysis results of Agm, Asp, bAla, Cys, Hcy, His, PEA, Phe, Ser, Spm, Syn, and TRA;

FIG. 7 illustrates results of confirming ratios of 10 neurochemicals that show significant differences depending on the presence or absence of stimulation in biological samples collected after 2 hours after stimulation; and

FIGS. 8A and 8B illustrate results of performing principal component analysis by combining ratios of neurochemicals in biological samples collected after 2 hours after stimulation.

### DETAILED DESCRIPTION

The present inventors conducted extensive research to establish a brain doping test for which a diagnostic method has not been studied, and completed the present disclosure.

The present inventors applied electrical stimulation to the brain by applying current using tDCS at a Cz location and measured brain waves. As a result, the present inventors confirmed that the electrical stimulation reduced the size of MRCP and that the reduction in the size of MRCP required less energy to perform a motion. In addition, as a result of the exercise evaluation performed before/after the electrical stimulation, it was confirmed that brain electrical stimulation improved the motor ability of a subject, and it was thus reconfirmed that brain doping improved motor ability as shown by existing research results.

A doping test ideally uses biological samples obtained in a minimal-invasive or non-invasive manner. Accordingly, the present inventors simultaneously analyzed neurotransmitters and metabolites thereof detected from urine samples using urine samples. Substances to be analyzed are a total of 36 neurotransmitters and metabolites thereof, including 23 substances that are target substances of KR10-2340107 and 13 novel analytes of Ade (Adenosine), Agm (Agmatine), Asp (Aspartic acid), bAla (β-alanine), Cys (Cysteine), Hcy (Homocysteine), His (Histamine), PEA (Phenethylamine), Phe (Phenylalanine), Ser (Serine), Spm (Spermine), Syn (Synephrine), and TRA (Tryptamine) (Table 1). The quantitative analysis validity of the novel analytes was verified through analysis of selectivity, linearity, accuracy, and detection limit (Table 3).

Neurochemicals, which were neurotransmitters and metabolites thereof detected in urine samples, showed significant changes before/after electrical stimulation, respectively, and to confirm the presence or absence of electrical stimulation based on the time point of diagnosis, the ratios of neurochemicals that showed significant differences depending on the presence or absence of stimulation were derived (Table 4). Accordingly, the present disclosure provides ratios of a total of 10 neurochemicals as biomarkers for confirming the presence or absence of brain electrical stimulation.

In the present disclosure, brain doping diagnosis provides information on whether a subject has a history of applying electrical stimulation to the brain before the diagnosis, and the diagnostic results indicate whether the subject has a history of receiving brain electrical stimulation or the possibility that the subject has received brain electrical stimulation.

The present disclosure may have various modifications and various Examples, and specific Examples will be hereinafter illustrated in the drawings and described in detail in the detailed description. However, the present disclosure is not limited to specific Embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Experiment Method

### 1. Induction of Brain Doping Through Current Stimulation

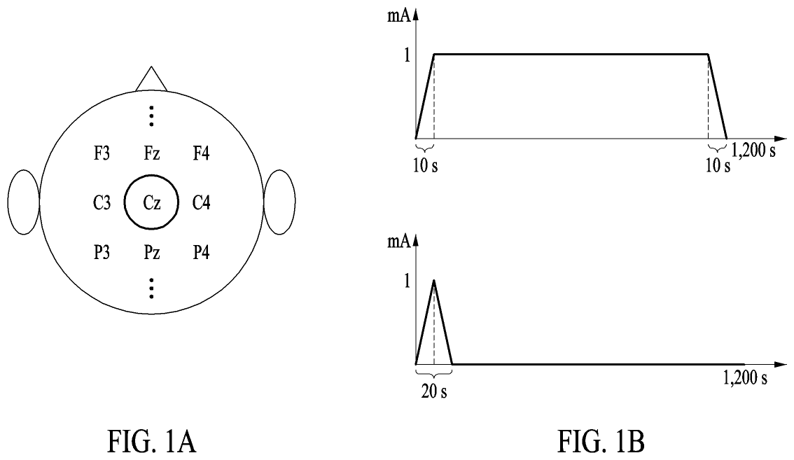

Brain stimulation protocols were designed and performed using NIC2 software provided by Neuroelectrics Co., Ltd.
Brain stimulation used transcranial direct current stimulation (tDCS). The tDCS was a non-invasive form of nerve stimulation to be transmitted through electrodes and was known to affect neural activity in a specific brain region by running a weak electric current to the scalp for a certain period of time.
In a brain stimulation location, an anode was selected as Cz, a primary motor cortex, and a cathode was selected as Fz according to the 10-20 international electroencephalography system (FIG. 1A).
The experiment was conducted twice with 15 Korean men in 20s and 30s at one-week intervals, and actual or sham stimulation was randomly applied for each session. The experiment was conducted without disclosing to subjects the presence or absence of stimulation.
The tDCS parameters used in the protocol were configured to apply stimulation for 20 minutes at an intensity of 1 mA, and allow direct current to flow from an anode electrode to a cathode electrode. Sham stimulation was applied for 20 minutes by ramping-up to the intensity of 1 mA for 10 seconds and then immediately ramping-down the intensity for 10 seconds (FIG. 1B).
tDCS equipment used for brain stimulation is Starstim 8 from Neuroelectrics, which enables tDCS stimulation and EEG measurement of a 8-channel. A NG Pistim (cross-sectional area: 3.14 cm²) with dual functions of stimulation and measurement was used.

### 2. Verification of Brain Doping Effect

2-1) Brain wave change pattern
To observe whether tDCS stimulation caused changes in brain waves, EEG measurement was performed a total of twice before and after brain stimulation. Since twice stimulations (tDCS and sham) were applied per subject, EEG measurement was performed a total of four times (measurement channels: Fpz, F3, F4, C3, Cz, C4, Pz, and Oz).
In order to identify motor-related changes in the measured EEG, the brain wave of interest was movement-related cortical potential (MRCP), which was a low-frequency signal (0 to 5 Hz) that occurred immediately before a voluntary movement of a person. It has the characteristic of an increasing negative slope immediately before approximately 500 ms from the start of movement, and the size and time of occurrence of MRCP are known to reflect motor ability.
For the MRCP measurement of the subject, the subject voluntarily performed 50 foot-taps per experiment using a foot pedal during EEG measurement.
For signal preprocessing, the brain waves at the Cz position were processed with a bandpass filter of 0.1 to 5 Hz.

### 2-2) Exercise Evaluation

Exercise evaluation after brain stimulation was performed on a treadmill by using respiratory gas measurement mask and analyzer (Quark CPET, COSMED, Italy). Additionally, a heart rate measurement device that was linked to the Quark CPET equipment was used.
Before performing the exercise, subjects were prepared by receiving instructions, attaching measurement masks, and removing the tops.
Exercise evaluation was conducted using the Bruce Protocol during a maximal exercise stress test. The protocol starts with an initial speed of 2.74 km/h and a 10% incline, and both the speed and the incline increase by 2% every 3 minutes and configured by a total of 7 stages and 21 minutes. Since the evaluation was terminated when the subject expressed the stop intention (all-out status), the subjects was instructed to perform the exercise as much as possible before starting.
As an exercise evaluation indicator, a maximal oxygen uptake (VO₂ max) is an indicator that shows the maximum value of oxygen uptake per unit time, reflecting the amplitude of energy produced in the muscles during exercise. In evaluation of athletes, high scores on the corresponding indicator are known as an indicator of excellent motor ability. In addition, the motor ability was evaluated using a run distance, energy consumption, and maximum heart rate as indicators.

### 3. Simultaneous Quantitative Analysis of 36 Neurochemicals in Biological Samples

### 3-1) Development of Analysis Method

Liquid chromatography-mass spectrometry (LC-MS) analysis was performed to 5 simultaneously analyze 36 neurochemicals in biological samples.
Substances to be analyzed were added with 13 novel substances of Ade (Adenosine), Agm (Agmatine), Asp (Aspartic acid), bAla (β-alanine), Cys (Cysteine), Hcy (Homocysteine), His (Histamine), PEA (Phenethylamine), Phe (Phenylalanine), Ser (Serine), Spm (Spermine), Syn (Synephrine), and TRA (Tryptamine) compared to prior Korean Patent (Simultaneous analysis method of neurotransmitters and metabolites from the same using derivatization, KR10-2340107), and the entire substances to be analyzed were as shown in Table 1 below.

TABLE 1

| No. | Substances |
|---|---|
| 1 | 3-methoxytyramine (3-MT) |
| 2 | 5-hydroxyindole-3-acetic acid (5-HIAA) |
| 3 | Serotonin (5-HT) |
| 4 | 5-hydroxytryptophan (5-HTP) |
| 5 | Adenosine (Ade) |
| 6 | Agmatine (Agm) |
| 7 | Aspartic acid (Asp) |
| 8 | β-alanine (bAla) |
| 9 | Cysteine (Cys) |
| 10 | Dopamine (DA) |
| 11 | 3,4-dihydroxyphenylglycol (DHPG) |
| 12 | 3,4-dihydroxyphenylacetic acid (DOPAC) |
| 13 | Epinephrine (EP) |
| 14 | γ-aminobutyric acid (GABA) |
| 15 | Glutamic acid (Glu) |
| 16 | Homocysteine (Hcy) |
| 17 | Histamine (His) |
| 18 | Homovanillicacid (HVA) |
| 19 | Kynurenine (Kyn) |
| 20 | 3,4-dihydroxy-1-phenylalanine (L-DOPA) |
| 21 | 4-hydroxy-3-methoxy-phenylglycol (MHPG) |
| 22 | Metanephrine (MN) |
| 23 | Norepinephrine (NE) |
| 24 | Normetanephrine (NMN) |
| 25 | Octopamine (OA) |
| 26 | Phenethylamine (PEA) |
| 27 | Phenylalanine (Phe) |
| 28 | Serine (Ser) |
| 29 | Spermine (Spm) |
| 30 | Synephrine (Syn) |
| 31 | Tyramine (TA) |
| 32 | Tryptamine (TRA) |
| 33 | Tryptophan (Trp) |
| 34 | Tyrosine (Tyr) |
| 35 | Vanillylmandelicacid (VMA) |
| 36 | Acetylcholine (ACh) |

A pretreatment process of the samples was performed in the same manner as the method of KR10-2340107.
The multiple reaction monitoring (MRM) conditions in the LC-MS equipment for detection of newly added substances were as shown in Table 2 below.

TABLE 2

| | | Precursor | Product | Collision | Retention |
|---|---|---|---|---|---|
| | | ion | ion | energy | time |
| Compounds | Adducts | (m/z) | (m/z) | (eV) | (min) |
| Ade | [M + H]⁺ | 340 | 136 | 12 | 1.87 |
| Agm | [M + H]⁺ | 233 | 98 | 17 | 2.07 |
| Asp | [M + H]⁺ | 262 | 142 | 16 | 2.33 |
| bAla | [M + H]⁺ | 190 | 102 | 12 | 2.06 |
| Cys | [M + H]⁺ | 294 | 176 | 10 | 2.77 |
| Hcy | [M + H]⁺ | 308 | 128 | 16 | 2.94 |
| His | [M + H]⁺ | 256 | 95 | 27 | 2.11 |
| PEA | [M + H]⁺ | 194 | 105 | 14 | 2.59 |
| Phe | [M + H]⁺ | 266 | 120 | 23 | 2.92 |
| Ser | [M + H]⁺ | 278 | 188 | 7 | 2.39 |
| Spm | [M + H]⁺ | 491 | 399 | 18 | 2.75 |
| Syn | [M + H]⁺ | 312 | 294 | 8 | 2.46 |
| TRA | [M + H]⁺ | 233 | 117 | 31 | 2.51 |

### 3-2) Validity Evaluation

To evaluate the validity of an analysis method with the addition of novel substances, selectivity, accuracy, linearity, and detection limit for 13 novel substances were evaluated.

### 4. Diagnosis Strategy for Brain Doping Through Changes in Neurochemicals

### 4-1) Analysis of Biological Samples

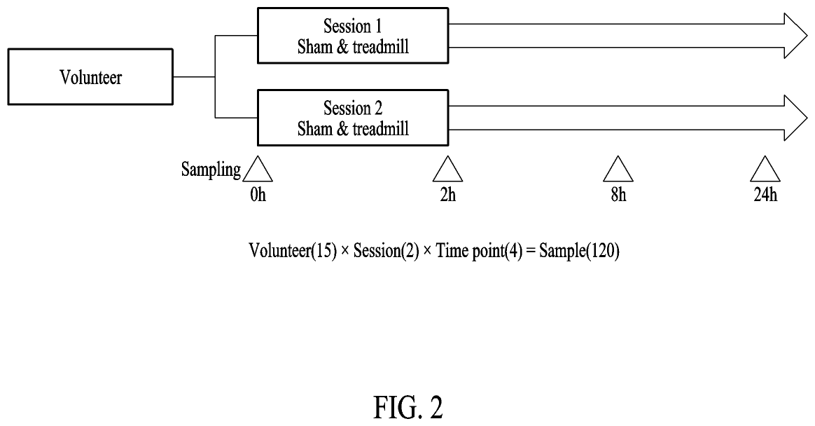

Urine samples were collected for each subject separately at pre-stimulation, 2 hours after stimulation, 8 hours after stimulation, and 24 hours after stimulation. As a rule, subjects participated twice for each stimulation, and accordingly, eight urine samples were obtained for each subject. The urine samples were freeze-stored until analysis to maintain sample stability (FIG. 2).
The collected samples were quantitatively analyzed according to the developed analysis method. To minimize variation between the samples, changes before and after brain stimulation were observed through a ratio of two different substances among substances to be analyzed.

### 4-2) Statistical Processing

Principal Component Analysis (PCA) is a statistical method that efficiently reduces high-dimensional data with a correlation between parameters into low-dimensional data while minimizing information loss, and the quantitative analysis values of all substances to be analyzed were used in PCA to confirm the comprehensive pattern differences between neurochemicals before and after stimulation, and to establish a brain doping diagnosis method using the same.

### Experimental Results

### 1. Exercise Evaluation

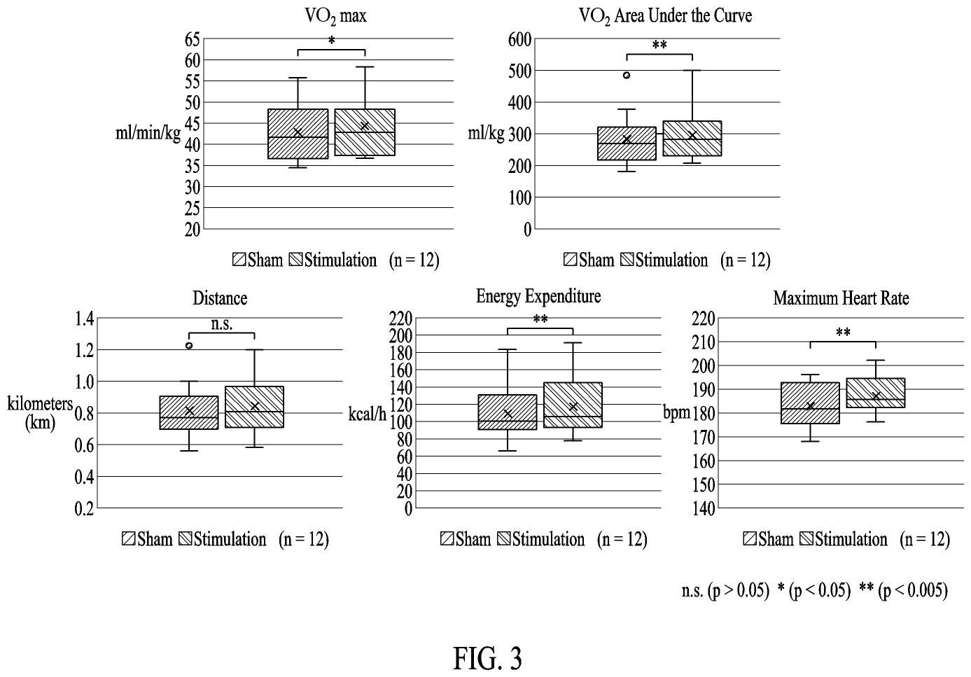

FIG. 3 illustrates results of exercise evaluation after electrical stimulation. Except for a run distance among the exercise evaluation indicators, a significant increased pattern was observed with just a single stimulation. Among the evaluation indicators, VO₂ Area Under the Curve indicates a total oxygen uptake.

### 2. Brain Wave Changes

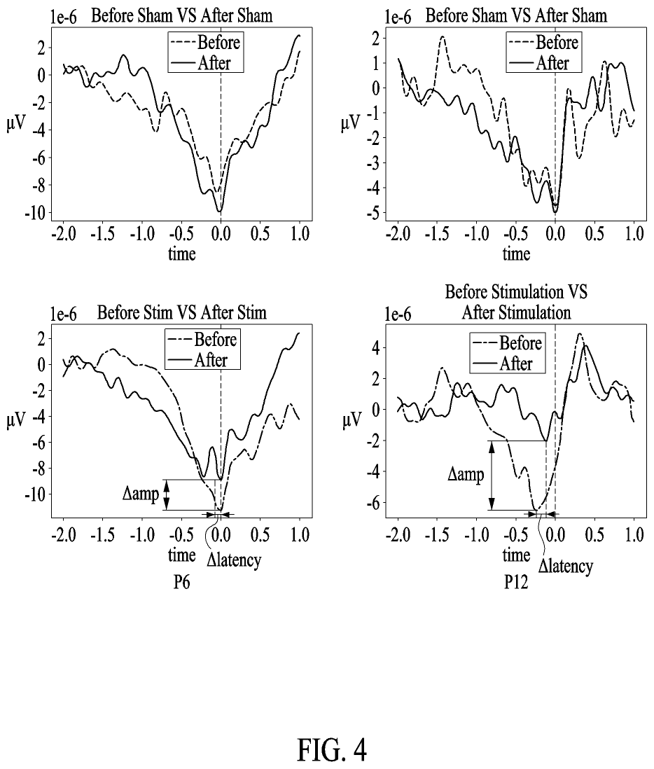

FIG. 4 illustrates results of measuring brain wave changes of Subjects 6 and 12 after electrical stimulation.

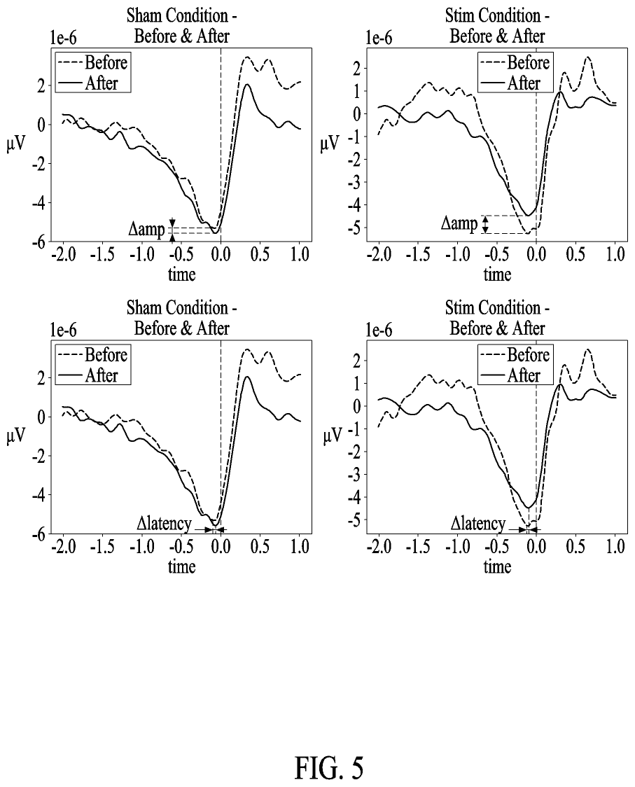

Based on the amplitude and latency of MRCP, in the case of Sham, there was no significant change before and after stimulation, but in the case of tDCS, it was confirmed that the amplitude of MRCP decreased and the onset time appeared earlier than before stimulation. From the above, it can be seen that brain stimulation reduces energy required to perform voluntary movement and a preparation time for performing movement.
Meanwhile, the results of brain wave measurement of all subjects, no significant change was not confirmed in the timing of MRCP occurrence after tDCS stimulation, but it was confirmed that the amplitude of MRCP was significantly reduced compared to a Sham group (FIG. 5).

### 3. Validity Evaluation of Neurochemical Analysis in Biological Samples

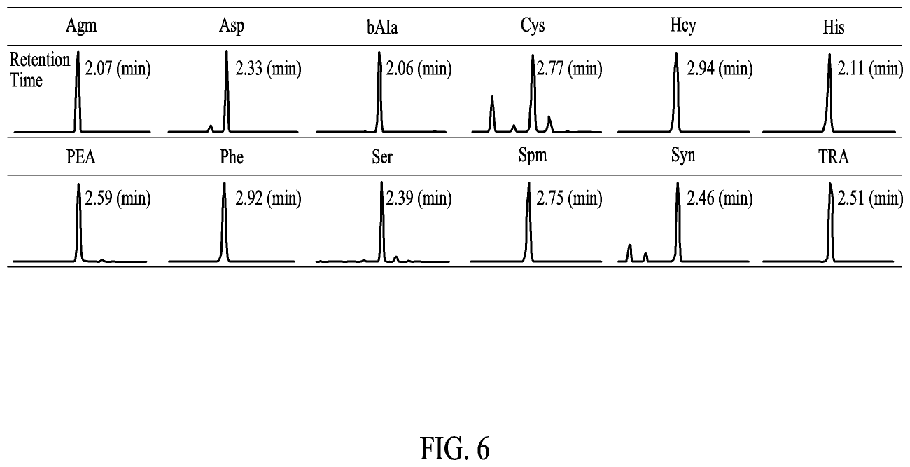

FIG. 6 illustrates LC-MS/MS analysis results of Agm, Asp, bAla, Cys, Hcy, His, PEA, Phe, Ser, Spm, Syn, and TRA.

Compared to prior Korean Patent No. 10-2340107, detection validity of 13 substances additionally analyzed in the present disclosure was confirmed as shown in Table 3 below.

TABLE 3

| | | | | | | | | Limit of |
|---|---|---|---|---|---|---|---|---|
| Com- | Calibration range | | | Linearity | Accuracy (%) | | | detection |
| pounds | (ng/mL) | | | (R²) | Low | Mid | High | (ng/mL) |
| Ade | 0.2 | — | 50 | 0.9998 | 103.3 | 105.6 | 98.7 | 0.1 |
| Agm | 0.5 | — | 200 | 0.9980 | 114.5 | 95.9 | 97.7 | 0.2 |
| Asp | 0.05 | — | 50 | 0.9999 | 108.8 | 91.5 | 100.2 | 0.02 |
| bAla | 0.2 | — | 200 | 0.9999 | 114.2 | 92.4 | 104.0 | 0.1 |
| Cys | 1 | — | 100 | 0.9976 | 111.0 | 97.9 | 101.4 | 0.5 |
| Hcy | 0.05 | — | 1 | 0.9999 | 95.2 | 93.7 | 106.3 | 0.02 |
| His | 0.005 | — | 5 | 0.9997 | 97.5 | 109.7 | 93.7 | 0.002 |
| PEA | 1 | — | 10 | 0.9981 | 103.1 | 99.4 | 98.0 | 0.5 |
| Phe | 0.1 | — | 200 | 0.9999 | 106.7 | 98.1 | 98.7 | 0.05 |
| Ser | 2 | — | 200 | 0.9987 | 111.7 | 102.5 | 102.4 | 1 |
| Spm | 0.5 | — | 20 | 0.9995 | 92.8 | 92.3 | 100.6 | 0.2 |
| Syn | 0.05 | — | 2 | 0.9999 | 103.0 | 94.9 | 105.0 | 0.02 |
| TRA | 0.01 | — | 5 | 0.9995 | 100.7 | 98.7 | 93.5 | 0.005 |

### 4. Diagnostic Criteria

### 1) Ratios Between Substances

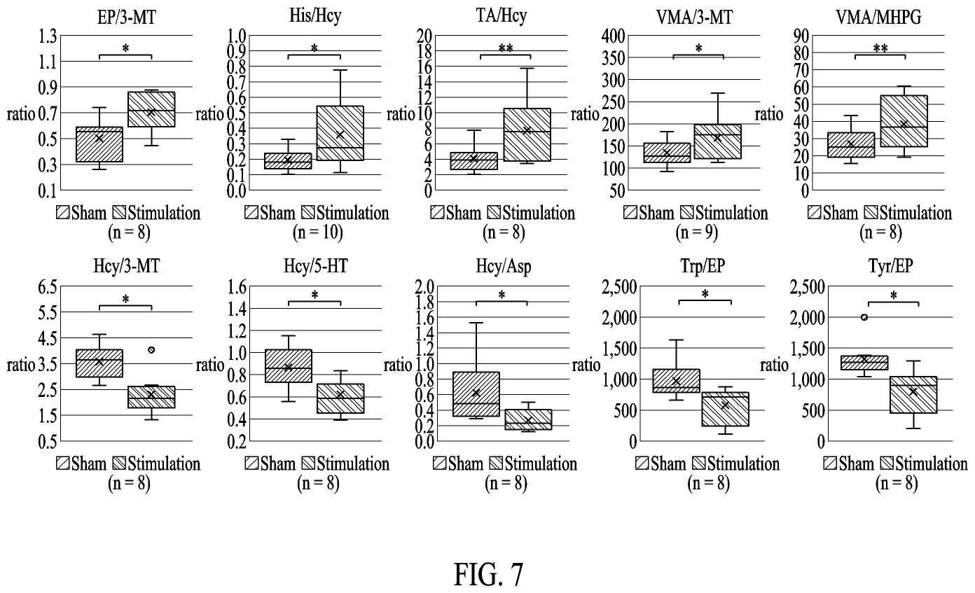

Ratios between 10 neurochemicals that showed significant differences depending on the presence or absence of stimulation were confirmed in biological samples collected after 2 hours after stimulation. The results were as shown in FIG. 7.
A strategy for diagnosing the presence or absence of brain stimulation based on a maximum or minimum value of a control group (sham) distribution range is presented in Table 4 below.

TABLE 4

| Substance ratio | EP/3-MT | His/Hcy | TA/Hcy | VMA/3-MT | VMA/MHPG |
|---|---|---|---|---|---|
| Standard | >0.74 | >3.27 | >7.72 | >182 | >43.3 |

| Substance ratio | Hcy/3-MT | Hcy/5-HT | Hcy/Asp | Trp/EP | Tyr/EP |
|---|---|---|---|---|---|
| Standard | <2.66 | <0.56 | <0.29 | <663 | <1036 |

For example, if EP/3-MT measured in urine samples of subjects is greater than 0.74, the subjects may be diagnosed as receiving brain stimulation.

### 2) PCA Pattern

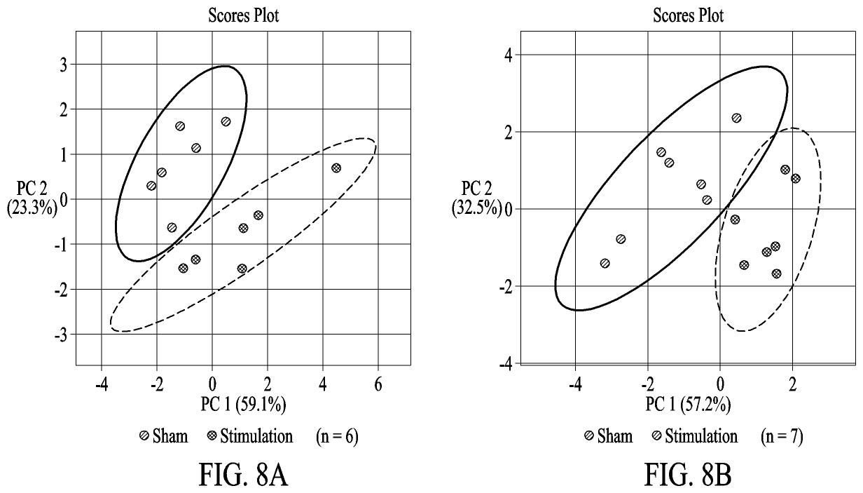

PCA was performed by combining the ratios of neurochemicals in biological samples collected 2 hours after stimulation, and differences in distribution patterns between the control group (sham) and actual stimulation (tDCS) were confirmed, and the results are shown in FIG. 8.
FIG. 8A illustrates an analysis result in which six substance ratios His/Hcy, TA/Hcy, VMA/3-MT, Hcy/3-MT, Hcy/5-HT, and Hcy/Asp were applied as parameters.
FIG. 8B illustrates an analysis result in which five substance ratios Hcy/3-MT, Hcy/5-HT, Hcy/Asp, Trp/EP, and Tyr/EP were applied as parameters.

As described above, although the embodiments have been described by the restricted drawings, various modifications and variations can be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result can be achieved.

Therefore, other implementations, other embodiments, and equivalents to the appended claims fall within the scope of the claims to be described below.

What is claimed is:

1. A biomarker composition for providing information on brain electrical stimulation comprising at least one indicator selected from a group consisting of a ratio of epinephrine to 3-methoxytyramine, a ratio of histamine to homocysteine, a ratio of tyramine to homocysteine, a ratio of vanillylmandelic acid to 3-methoxytyramine, a ratio of vanillylmandelic acid to 4-hydroxy-3-methoxy-phenylglycol, a ratio of homocysteine to 3-methoxytyramine, a ratio of homocysteine to serotonin, a ratio of homocysteine to aspartic acid, a ratio of tryptophan to epinephrine, and a ratio of tyrosine to epinephrine in urine.

2. The biomarker composition for providing information on brain electrical stimulation of claim 1, wherein the composition is used to predict whether there is a history of electrical stimulation applied to the brain to improve motor ability.

3. A method for providing information for brain doping diagnosis comprising the following:
(A) obtaining a urine sample from a subject;
(B) preparing a neurochemical profile by measuring contents of neurotransmitters and metabolites thereof in the sample; and
(C) calculating one or more indicators selected from a group consisting of a ratio of epinephrine to 3-methoxytyramine (EP/3-MT), a ratio of histamine to homocysteine (His/Hcy), a ratio of tyramine to homocysteine (TA/Hcy), a ratio of vanillylmandelic acid to 3-methoxytyramine (VMA/3-MT), a ratio of vanillylmandelic acid to 4-hydroxy-3-methoxy-phenylglycol (VMA/MHPG), a ratio of homocysteine to 3-methoxytyramine (Hcy/3-MT), a ratio of homocysteine to serotonin (Hcy/5-HT), a ratio of homocysteine to aspartic acid (Hcy/Asp), a ratio of tryptophan to epinephrine (Trp/EP), and a ratio of tyrosine to epinephrine (Tyr/EP) from the profile.

4. The method for providing information for brain doping diagnosis of claim 3, wherein (B) is performed through liquid chromatography-mass spectrometry.

5. The method for providing information for brain doping diagnosis of claim 3, further comprising:
(D) determining that the subject has a history of receiving electrical stimulation to the brain or has a high possibility thereof in the case of one or more of the following conditions (1) to (10), after (C):
(1) when the ratio of epinephrine to 3-methoxytyramine (EP/3-MT) is greater than 0.74
(2) when the ratio of histamine to homocysteine (His/Hcy) is greater than 3.27
(3) when the ratio of tyramine to homocysteine (TA/Hcy) is greater than 7.72
(4) when the ratio of vanillylmandelic acid to 3-methoxytyramine (VMA/3-MT) is greater than 182
(5) when the ratio of vanillylmandelic acid to 4-hydroxy-3-methoxy-phenylglycol (VMA/MHPG) is greater than 43.3
(6) when the ratio of homocysteine to 3-methoxytyramine (Hcy/3-MT) is less than 2.66
(7) when the ratio of homocysteine to serotonin (Hcy/5-HT) is less than 0.56
(8) when the ratio of homocysteine to aspartic acid (Hcy/Asp) is less than 0.29
(9) when the ratio of tryptophan to epinephrine (Trp/EP) is less than 663
(10) when the ratio of tyrosine to epinephrine (Tyr/EP) is less than 1036.
